(19) United States
(12) Patent Application Publication
(10) Pub. No.: US 2026/0207566 A1
(43) Pub. Date: Jul. 23, 2026

(54) COMPOUNDS FOR TREATMENT OF HAIR LOSS

(71) Applicant: PELAGE PHARMACEUTICALS, INC., Los Angeles, CA (US)

(72) Inventors: Daniel Lui Sun, Cypress, CA (US); Daniel Walter Gil, Corona Del Mar, CA (US)

(21) Appl. No.: 19/144,428

(22) PCT Filed: Dec. 27, 2023
(86) PCT No.: PCT/US2023/086052

§ 371 Date: Jun. 27, 2025

Related U.S. Application Data
(60) Provisional application No. 63/477,347, filed on Dec. 27, 2022.

(51) Int. Cl. A61K 31/437; C07D 471/04

(52) U.S. Cl. CPC A61K 31/437; C07D 471/04

(57) ABSTRACT

Compounds represented by Formula 1, as depicted herein, are described in this disclosure. Use, methods, and medicaments related to treatment of conditions or diseases, such as for growing hair in a mammal, are also described herein.

### CROSS-REFERENCE TO RELATED APPLICATIONS

This application is a National Stage of International Application No. PCT/US2023/086052, filed Dec. 27, 2023, which claims priority to U.S. Provisional Application No. 63/477,347, filed Dec. 27, 2022, each of which are hereby incorporated by reference in their entireties.

### BACKGROUND

Hair follicle stem cells (HFSCs) undergo successive rounds of quiescence (telogen) punctuated by brief periods of proliferation correlating with the start of the hair cycle (telogen-anagen transition). Proliferation or activation of HFSCs is well known to be a prerequisite for advancement of the hair cycle. Despite advances in treatment options, baldness and alopecia continue to be conditions that cannot be successfully treated in many individuals. Some of the existing treatments are inconvenient for users, others require surgical intervention or other invasive procedures. Additional therapies are needed.

### SUMMARY

Some embodiments include a compound represented by Formula 1:

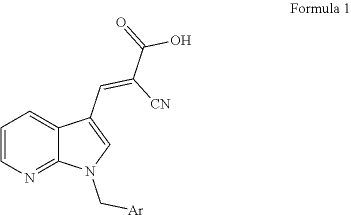

or an E/Z isomer thereof; wherein Ar is optionally substituted aryl, such as optionally substituted phenyl or optionally substituted naphthyl.

Some embodiments include a pharmaceutical composition comprising a compound described herein. In some embodiments, the pharmaceutical composition is for treating a disease or a disorder, such as a disease or disorder affecting or related to hair growth.

Some embodiments include a pharmaceutical composition for growing hair comprising a compound described herein.

Some embodiments include a method of growing hair, comprising: administering a compound described herein to the skin of a mammal in the area where hair growth is intended.

Some embodiments include use of a compound described herein in the manufacture of a medicament for growing hair.

Some embodiments include use of a compound described herein in the manufacture of a medicament for treating a disease or disorder.

Some embodiments include a method of growing hair comprising administering a compound described herein to a mammal in need thereof.

Some embodiments include a method of treating a disease or disorder, such as a disorder affecting hair growth comprising administering a compound described herein to a mammal in need thereof. In some embodiments, the disorder is alopecia or baldness.

Some embodiments include a kit comprising a compound described herein and a label with instructions to administer the compound for a use described herein, such as growing hair.

### DETAILED DESCRIPTION

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; deuterium-modified forms; Z and E olefin isomers; tautomers; or any other chemical species that may rapidly convert to a compound described herein under conditions in which the compounds are used as described herein. In some embodiments, the compound contains more than a natural abundance of deuterium. In some embodiments, one or more of the hydrogen atoms on the compound is replaced by deuterium so that the compound is at least 50%, at least 80%, at least 90%, at least 95%, or at least 99% deuterium in that position. The E/Z isomers of any structure depicted herein are specifically contemplated.

Unless otherwise indicated, when a compound or chemical structural feature (such as alkyl or aryl) is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of about 15 g/mol to about 50 g/mol, about 15 g/mol to about 100 g/mol, about 15 g/mol to about 150 g/mol, about 15 g/mol to about 200 g/mol, about 15 g/mol to about 300 g/mol, or about 15 g/mol to about 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes one C, N, O, S, P, Si, F, Cl, Br, or I atom. Examples of substituents include, but are not limited to, compounds represented by an empirical formula: C₁₋₁₂H₃₋₂₉O₀₋₄N₀₋₄S₀₋₄F₀₋₂₅Cl₀₋₅Si₀₋₃P₀₋₃, C₀₋₁₂H₀₋₂₉O₁₋₄N₀₋₄S₀₋₄F₀₋₂₅Cl₀₋₅Si₀₋₃P₀₋₃, C₀₋₁₂H₀₋₂₉O₀₋₄N₁₋₄S₀₋₄F₀₋₂₅Cl₀₋₅Si₀₋₃P₀₋₃, C₀₋₁₂H₀₋₂₉O₀₋₄N₀₋₄S₁₋₄F₀₋₂₅Cl₀₋₅Si₀₋₃P₀₋₃, C₀₋₁₂H₀₋₂₉O₀₋₄N₀₋₄S₀₋₄F₁₋₂₅Cl₀₋₅Si₀₋₃P₀₋₃, C₀₋₁₂H₀₋₂₉O₀₋₄N₀₋₄S₀₋₄F₀₋₂₅Cl₁₋₅Si₀₋₃P₀₋₃, C₀₋₁₂H₀₋₂₉O₀₋₄N₀₋₄S₀₋₄F₀₋₂₅Cl₀₋₅Si₁₋₃P₀₋₃, C₀₋₁₂H₀₋₂₉O₀₋₄N₀₋₄S₀₋₄F₀₋₂₅Cl₀₋₅Si₀₋₃P₁₋₃, C₁₋₆H₃₋₁₆O₀₋₄N₀₋₄S₀₋₄F₀₋₁₃Cl₀₋₃Si₀₋₃P₀₋₃, C₀₋₆H₀₋₁₆O₁₋₄N₀₋₄S₀₋₄F₀₋₁₃Cl₀₋₃Si₀₋₃P₀₋₃, C₀₋₆H₀₋₁₇O₀₋₄N₁₋₄S₀₋₄F₀₋₁₃Cl₀₋₃Si₀₋₃P₀₋₃, C₀₋₆H_{0- 17}O₀₋₄N₀₋₄S₁₋₄F₀₋₁₃Cl₀₋₃Si₀₋₃P₀₋₃, C₀₋₆H₀₋₁₇O₀₋₄N₀₋₄S₀₋₄F₁₋₁₃Cl₀₋₃Si₀₋₃P₀₋₃, C₀₋₆H₀₋₁₇O₀₋₄N₀₋₄S₀₋₄F₀₋₁₃Cl₁₋₃Si₀₋₃P₀₋₃, C₀₋₆H₀₋₁₇O₀₋₄N₀₋₄S₀₋₄F₀₋₁₃Cl₀₋₃Si₁₋₃P₀₋₃, C₀₋₆H₀₋₁₇O₀₋₄N₀₋₄S₀₋₄F₀₋₁₃Cl₀₋₃Si₀₋₃P₁₋₃, C₁₋₁₂H₃₋₂₉O₀₋₄N₀₋₄S₀₋₄F₀₋₂₅Cl₀₋₅P₀₋₃, C₁₋₁₂H₃₋₂₇O₀₋₄N₀₋₂S₀₋₂F₀₋₂₅Cl₀₋₅P₀₋₁, C₁₋₁₂H₃₋₂₇O₀₋₄N₀₋₂, C₁₋₁₂H₃₋₂₅O₀₋₄, C₁₋₁₂H₃₋₂₇N₀₋₂, C₁₋₉H₃₋₂₁O₀₋₄N₀₋₂S₀₋₂F₀₋₁₉Cl₀₋₆P₀₋₁, C₁₋₉H₃₋₁₉F₀₋₁₉, C₁₋₉H₃₋₂₁O₀₋₄N₀₋₂, C₁₋₉H₃₋₁₉O₀₋₄, C₁₋₉H₃₋₂₁N₀₋₂, C₁₋₆H₃₋₁₅O₀₋₃N₀₋₂S₀₋₂F₀₋₁₃Cl₀₋₅P₀₋₁, C₁₋₆H₃₋₁₃F₀₋₁₃, C₁₋₆H₃₋₁₅O₀₋₄N₀₋₂, C₁₋₆H₃₋₁₃O₀₋₄, C₁₋₆H₃₋₁₅N₀₋₂, C₁₋₃H₃₋₉O₀₋₃N₀₋₂S₀₋₂F₀₋₁₃Cl₀₋₃P₀₋₁, C₁₋₃H₃₋₇F₀₋₇, C₁₋₃H₃₋₉O₀₋₃N_{0- 2}, C₁₋₃H₃₋₇O₀₋₃, C₁₋₃H₃₋₉N₀₋₂, F, Cl, Br, I, OH, OR^{A}, SH, SR^{A}, NH₂, NHRA, NR^{A}R^{B}, CF₃, CN, carboxylic acid, optionally substituted carboxylic ester, or optionally substituted C₁₋₆ alkyl, such as optionally substituted branched C₂₋₆ alkyl or optionally substituted linear C₁₋₆ alkyl, including optionally substituted branched or linear C₁₋₃ alkyl (e.g. —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, cyclopropyl, cyclobutyl, cyclopentyl, cyclohexyl, etc.), alkenyl, alkynyl, heteroalkyl, heteroalkenyl, heteroalkynyl, aryl, heteroaryl, carbocycle, heterocycle, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, etc.

For convenience, the term “molecular weight” is used with respect to a moiety or part of a molecule to indicate the sum of the atomic masses of the atoms in the moiety or part of a molecule, even though it may not be a complete molecule.

With respect to Formula 1, Ar is optionally substituted aryl, such as phenyl substituted with 0, 1, 2, 3, 4, or 5 substituents, or napthyl substituted with 0, 1, 2, 3, 4, 5, 6, or 7 substituents, the substituents for aryl, such as phenyl or naphthyl, may be any suitable substituents, such as F, Cl, Br, I, OH, CN, CF₃, —SF₆, C₁₋₆ alkyl (such as —CH₃, —C₂H₅, —C₃H₇, —C₄H₉, —C₅H₁₁, —C₆H₁₃, etc.), C₁₋₆O-alkyl (such as —O—CH₃, —O—C₂H₅, —O—C₃H₇, —O—C₄H₉, —O—C₅H₁₁, —O—C₆H₁₃, etc.), or C₁₋₆H₃₋₁₃O (such as —C(═O)CH₃ or —CH₂OCH₃).

With respect to Formula 1, in some embodiments, a phenyl group may have 0, 1, 2, 3, 4, or 5 substituents such as —OCH₃, —CF₃, —CH₃, —OCF₃, —SCF₃, —Cl, —SF₅, etc. In some embodiments, Ar is phenyl with 1 or 2 substituents which are independently —OCH₃, —CF₃, —CH₃, —OCF₃, —SCF₃, Cl, or —SF₅.

In some embodiments, Ar is phenyl with 1 substituent which is-CF₃, —CH₃, —OCF₃, —SCF₃, Cl, or —SF₅. In some embodiments, Ar is phenyl with 1 substituent which is-OCH₃, —CH₃, —OCF₃, —SCF₃, Cl, or —SF₅. In some embodiments, Ar is phenyl with 1 substituent which is-OCH₃, —CF₃, —OCF₃, —SCF₃, Cl, or —SF₅. In some embodiments, Ar is phenyl with 1 substituent which is-OCH₃, —CF₃, —CH₃, —SCF₃, Cl, or —SF₅.

In some embodiments, Ar is phenyl with 1 substituent which is-OCH₃, —CF₃, —CH₃, —OCF₃, —Cl, or —SF₅. In some embodiments, Ar is phenyl with 1 substituent which is-OCH₃, —CF₃, —CH₃, —OCF₃, —SCF₃, or —SF₅. In some embodiments, Ar is phenyl with 1 substituent which is-OCH₃, —CF₃, —CH₃, —OCF₃, —SCF₃, or Cl.

In some embodiments, Ar is phenyl with 2 substituents which are independently —CF₃, —CH₃, —OCF₃, —SCF₃, Cl, or —SF₅. In some embodiments, Ar is phenyl with 2 substituents which are independently —OCH₃, —CH₃, —OCF₃, —SCF₃, Cl, or —SF₅. In some embodiments, Ar is phenyl with 2 substituents which are independently —OCH₃, —CF₃, —OCF₃, —SCF₃, Cl, or —SF₅. In some embodiments, Ar is phenyl with 2 substituents which are independently —OCH₃, —CF₃, —CH₃, —SCF₃, Cl, or —SF₅. In some embodiments, Ar is phenyl with 2 substituents which are independently —OCH₃, —CF₃, —CH₃, —OCF₃, —Cl, or —SF₅. In some embodiments, Ar is phenyl with 2 substituents which are independently —OCH₃, —CF₃, —CH₃, —OCF₃, —SCF₃, or —SF₅. In some embodiments, Ar is phenyl with 2 substituents which are independently —OCH₃, —CF₃, —CH₃, —OCF₃, —SCF₃, or Cl.

In some embodiments, Ar is unsubstituted phenyl. In some embodiments, Ar is unsubstituted naphthyl.

In some embodiments, Ar is:

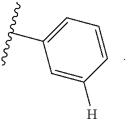 [SMILES: *c1ccccc1]

In some embodiments, Ar is:

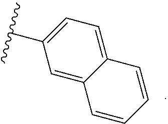 [SMILES: *c1ccc2ccccc2c1]

In some embodiments, Ar is:

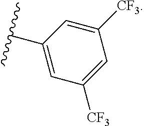 [SMILES: *c1cc(C(F)(F)F)cc(C(F)(F)F)c1]

In some embodiments, Ar is:

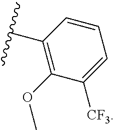 [SMILES: *c1cccc(C(F)(F)F)c1OC]

In some embodiments, Ar is:

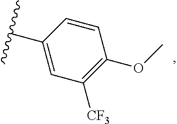 [SMILES: *c1ccc(OC)c(C(F)(F)F)c1]

In some embodiments, Ar is:

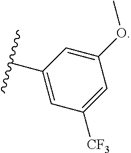 [SMILES: *c1cc(OC)cc(C(F)(F)F)c1]

In some embodiments, Ar is:

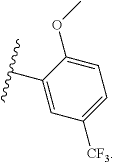 [SMILES: *c1cc(C(F)(F)F)ccc1OC]

In some embodiments, Ar is:

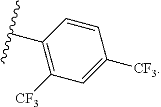 [SMILES: *c1ccc(C(F)(F)F)cc1C(F)(F)F]

In some embodiments, Ar is:

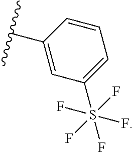 [SMILES: *c1cccc(S(F)(F)(F)(F)F)c1]

In some embodiments, Ar is:

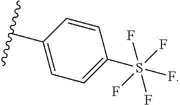 [SMILES: *c1ccc(S(F)(F)(F)(F)F)cc1]

In some embodiments, Ar is:

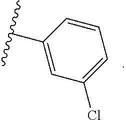 [SMILES: *c1cccc(Cl)c1]

In some embodiments, Ar is:

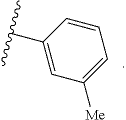 [SMILES: *c1cccc(C)c1]

In some embodiments, Ar is:

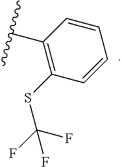 [SMILES: *c1ccccc1SC(F)(F)F]

In some embodiments, Ar is:

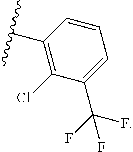 [SMILES: *c1cccc(C(F)(F)F)c1Cl]

In some embodiments, Ar is:

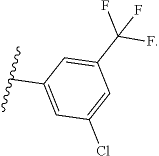 [SMILES: *c1cc(Cl)cc(C(F)(F)F)c1]

In some embodiments, Ar is:

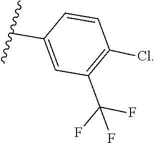 [SMILES: *c1ccc(Cl)c(C(F)(F)F)c1]

In some embodiments, Ar is:

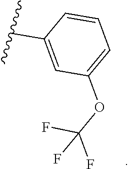 [SMILES: *c1cccc(OC(F)(F)F)c1]

In some embodiments, Ar is: CI

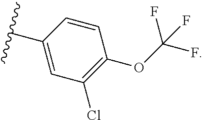 [SMILES: *c1ccc(OC(F)(F)F)c(Cl)c1]

In some embodiments, Ar is not:

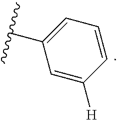 [SMILES: *c1ccccc1]

In some embodiments, Ar is not:

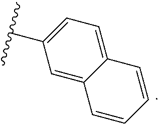 [SMILES: *c1ccc2ccccc2c1]

In some embodiments, Ar is not:

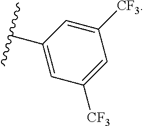 [SMILES: *c1cc(C(F)(F)F)cc(C(F)(F)F)c1]

In some embodiments, Ar is not:

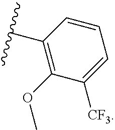 [SMILES: *c1cccc(C(F)(F)F)c1OC]

In some embodiments, Ar is not:

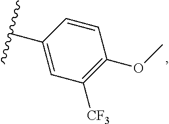 [SMILES: *c1ccc(OC)c(C(F)(F)F)c1]

In some embodiments, Ar is not:

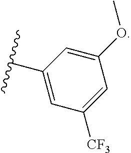 [SMILES: *c1cc(OC)cc(C(F)(F)F)c1]

In some embodiments, Ar is not:

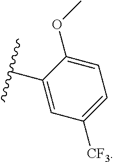 [SMILES: *c1cc(C(F)(F)F)ccc1OC]

In some embodiments, Ar is not:

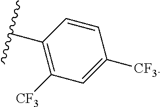 [SMILES: *c1ccc(C(F)(F)F)cc1C(F)(F)F]

In some embodiments, Ar is not:

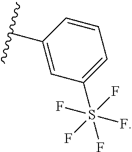 [SMILES: *c1cccc(S(F)(F)(F)(F)F)c1]

In some embodiments, Ar is not:

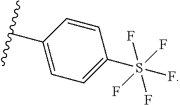 [SMILES: *c1ccc(S(F)(F)(F)(F)F)cc1]

In some embodiments, Ar is not:

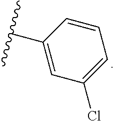 [SMILES: *c1cccc(Cl)c1]

In some embodiments, Ar is not:

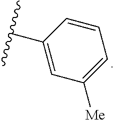 [SMILES: *c1cccc(C)c1]

In some embodiments, Ar is not:

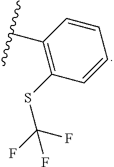 [SMILES: *c1ccccc1SC(F)(F)F]

In some embodiments, Ar is not:

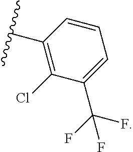 [SMILES: *c1cccc(C(F)(F)F)c1Cl]

In some embodiments, Ar is not:

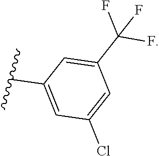 [SMILES: *c1cc(Cl)cc(C(F)(F)F)c1]

In some embodiments, Ar is not:

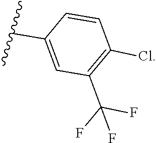 [SMILES: *c1ccc(Cl)c(C(F)(F)F)c1]

In some embodiments, Ar is not:

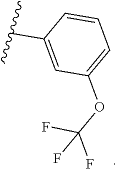 [SMILES: *c1cccc(OC(F)(F)F)c1]

In some embodiments, Ar is not:

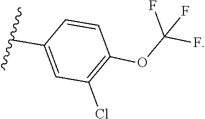 [SMILES: *c1ccc(OC(F)(F)F)c(Cl)c1]

In some embodiments, the compound is:

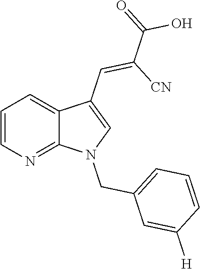 [SMILES: N#C/C(=C\c1cn(Cc2ccccc2)c2ncccc12)C(=O)O]

In some embodiments, the compound is:

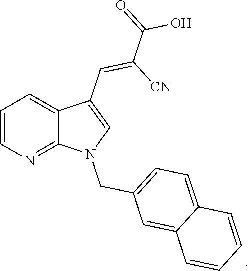 [SMILES: N#C/C(=C\c1cn(Cc2ccc3ccccc3c2)c2ncccc12)C(=O)O]

In some embodiments, the compound is not:

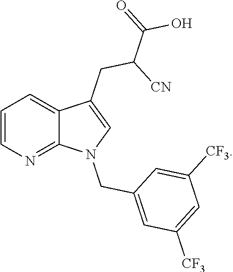 [SMILES: N#CC(Cc1cn(Cc2cc(C(F)(F)F)cc(C(F)(F)F)c2)c2ncccc12)C(=O)O]

In some embodiments, the compound is:

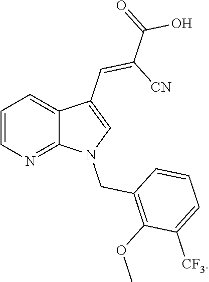 [SMILES: COc1c(Cn2cc(/C=C(\C#N)C(=O)O)c3cccnc32)cccc1C(F)(F)F]

In some embodiments, the compound is:

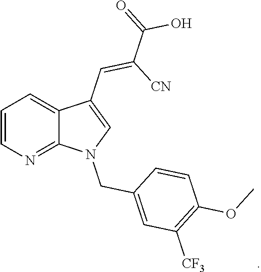 [SMILES: COc1ccc(Cn2cc(/C=C(\C#N)C(=O)O)c3cccnc32)cc1C(F)(F)F]

In some embodiments, the compound is:

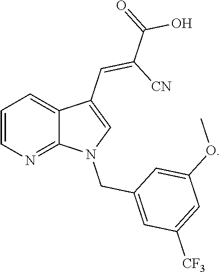 [SMILES: COc1cc(Cn2cc(/C=C(\C#N)C(=O)O)c3cccnc32)cc(C(F)(F)F)c1]

In some embodiments, the compound is:

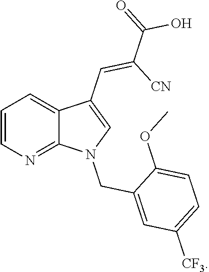 [SMILES: COc1ccc(C(F)(F)F)cc1Cn1cc(/C=C(\C#N)C(=O)O)c2cccnc21]

In some embodiments, the compound is:

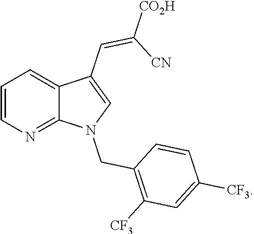 [SMILES: N#C/C(=C\c1cn(Cc2ccc(C(F)(F)F)cc2C(F)(F)F)c2ncccc12)C(=O)O]

In some embodiments, the compound is:

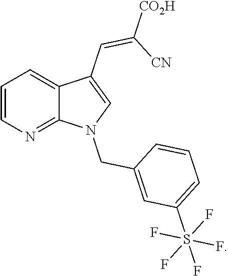 [SMILES: N#C/C(=C\c1cn(Cc2cccc(S(F)(F)(F)(F)F)c2)c2ncccc12)C(=O)O]

In some embodiments, the compound is:

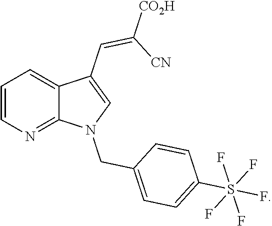 [SMILES: N#C/C(=C\c1cn(Cc2ccc(S(F)(F)(F)(F)F)cc2)c2ncccc12)C(=O)O]

In some embodiments, the compound is:

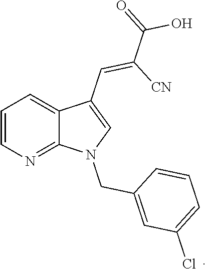 [SMILES: N#C/C(=C\c1cn(Cc2cccc(Cl)c2)c2ncccc12)C(=O)O]

In some embodiments, the compound is: Me

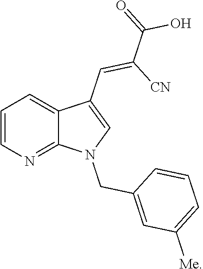 [SMILES: Cc1cccc(Cn2cc(/C=C(\C#N)C(=O)O)c3cccnc32)c1]

In some embodiments, the compound is:

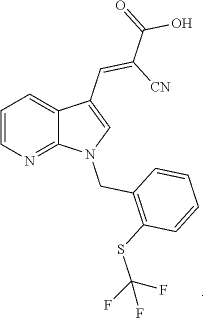 [SMILES: N#C/C(=C\c1cn(Cc2ccccc2SC(F)(F)F)c2ncccc12)C(=O)O]

In some embodiments, the compound is:

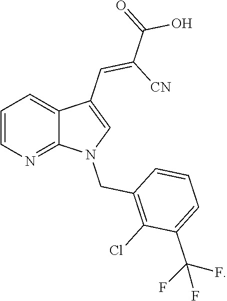 [SMILES: N#C/C(=C\c1cn(Cc2cccc(C(F)(F)F)c2Cl)c2ncccc12)C(=O)O]

In some embodiments, the compound is:

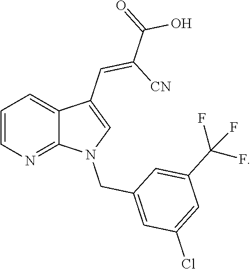 [SMILES: N#C/C(=C\c1cn(Cc2cc(Cl)cc(C(F)(F)F)c2)c2ncccc12)C(=O)O]

In some embodiments, the compound is:

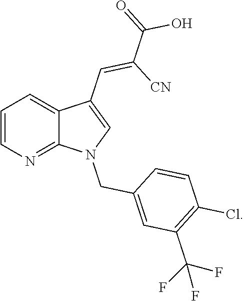 [SMILES: N#C/C(=C\c1cn(Cc2ccc(Cl)c(C(F)(F)F)c2)c2ncccc12)C(=O)O]

In some embodiments, the compound is:

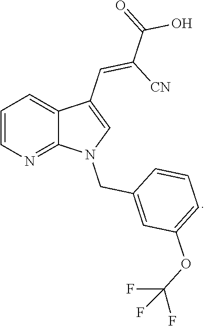 [SMILES: N#C/C(=C\c1cn(Cc2cccc(OC(F)(F)F)c2)c2ncccc12)C(=O)O]

In some embodiments, the compound is:

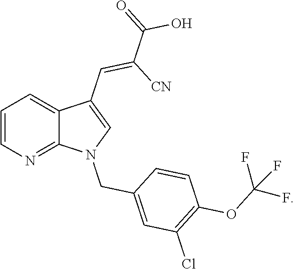 [SMILES: N#C/C(=C\c1cn(Cc2ccc(OC(F)(F)F)c(Cl)c2)c2ncccc12)C(=O)O]

In some embodiments, the compound is not:

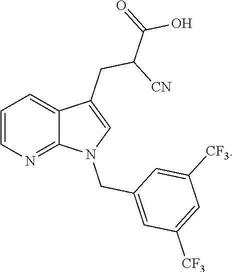 [SMILES: N#CC(Cc1cn(Cc2cc(C(F)(F)F)cc(C(F)(F)F)c2)c2ncccc12)C(=O)O]

The compounds described herein are useful for growing hair. For example, a compound described herein may be administered to the skin of a mammal in the area where hair growth is intended.

In certain aspects, the compounds of the present disclosure are mitochondrial pyruvate oxidation (MPO) inhibitors. In some embodiments, the compounds described herein may inhibit mitochondrial pyruvate carrier (MPC). In certain embodiments, the MPO inhibitor is an MPC inhibitor. In some aspects, inhibiting MPO in a cell has the effect of enhancing lactate production in a cell and/or enhancing the activity of lactic acid dehydrogenase (LDH) in a cell, and promoting hair growth. In certain aspects, the present disclosure provides methods of promoting hair growth or treating a hair growth condition or disorder such as baldness or alopecia, comprising administering to a patient an MPO inhibitor (e.g., topically, such as with a pharmaceutical composition formulated for topical application), such as a compound of the present disclosure. In certain embodiments, the present disclosure provides methods of promoting hair growth or treating a hair growth condition or disorder such as baldness or alopecia, comprising administering to a patient an MPC inhibitor (e.g., topically, such as with a pharmaceutical composition formulated for topical application), such as a compound of the present disclosure. In some embodiments, inhibiting the MPO or the MPC in a cell has the effect of enhancing lactate production and/or enhancing the activity of LDH in a cell, and promoting hair growth.

For the purposes of this disclosure, the term “treat,” “treating,” or a similar term (such as “modulating”), includes cure, mitigation, treatment, or prevention of disease in man or other animals, or any other effect that would be associated with a “drug” as defined under 21 USC 321(g).

The compounds below were prepared by adapting the method described in PCT/US21/39501, filed Jun. 29, 2021 entitled “COMPOSITIONS AND METHODS FOR MODULATING HAIR GROWTH,” Attorney Docket Number UCH-22825 (32246.22825)(see Example 5, pp. 73-75). FTIR and NMR analysis confirmed the structure.

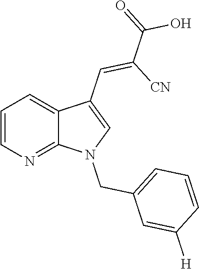 [SMILES: N#C/C(=C\c1cn(Cc2ccccc2)c2ncccc12)C(=O)O]

### (E)-3-(1-benzyl-1H-pyrrolo[2,3-b]pyridin-3-yl)-2-cyanoacrylic acid

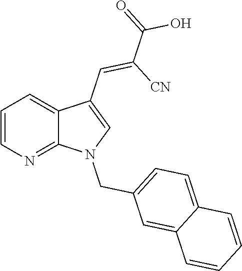 [SMILES: N#C/C(=C\c1cn(Cc2ccc3ccccc3c2)c2ncccc12)C(=O)O]

### (E)-2-cyano-3-(1-(naphthalen-2-ylmethyl)-1H-pyrrolo[2,3-b]pyridin-3-yl)acrylic acid

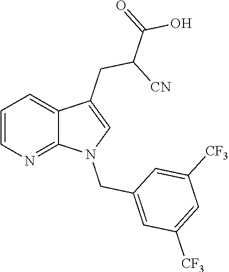 [SMILES: N#CC(Cc1cn(Cc2cc(C(F)(F)F)cc(C(F)(F)F)c2)c2ncccc12)C(=O)O]

### 3-(1-(3,5-bis(trifluoromethyl)benzyl)-1H-pyrrolo[2,3-b]pyridin-3-yl)-2-cyanopropanoic acid

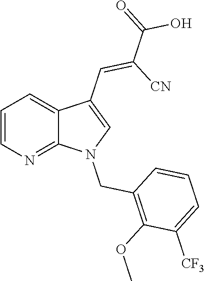 [SMILES: COc1c(Cn2cc(/C=C(\C#N)C(=O)O)c3cccnc32)cccc1C(F)(F)F]

### (E)-2-cyano-3-(1-(2-methoxy-3-(trifluoromethyl)benzyl)-1H-pyrrolo[2,3-b]pyridin-3-yl)acrylic acid

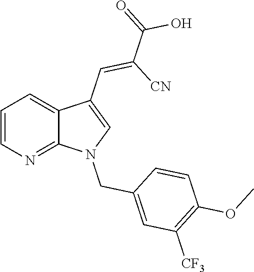 [SMILES: COc1ccc(Cn2cc(/C=C(\C#N)C(=O)O)c3cccnc32)cc1C(F)(F)F]

### (E)-2-cyano-3-(1-(4-methoxy-3-(trifluoromethyl)benzyl)-1H-pyrrolo[2,3-b]pyridin-3-yl)acrylic acid

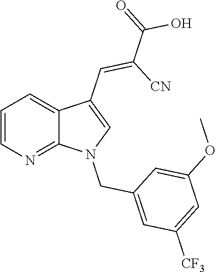 [SMILES: COc1cc(Cn2cc(/C=C(\C#N)C(=O)O)c3cccnc32)cc(C(F)(F)F)c1]

### (E)-2-cyano-3-(1-(3-methoxy-5-(trifluoromethyl)benzyl)-1H-pyrrolo[2,3-b]pyridin-3-yl)acrylic acid

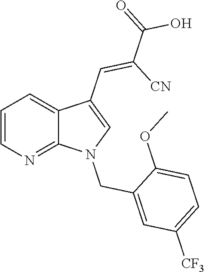 [SMILES: COc1ccc(C(F)(F)F)cc1Cn1cc(/C=C(\C#N)C(=O)O)c2cccnc21]

### (E)-2-cyano-3-(1-(2-methoxy-5-(trifluoromethyl)benzyl)-1H-pyrrolo[2,3-b]pyridin-3-yl)acrylic acid

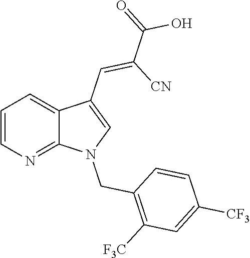 [SMILES: N#C/C(=C\c1cn(Cc2ccc(C(F)(F)F)cc2C(F)(F)F)c2ncccc12)C(=O)O]

### (E)-3-(1-(2,4-bis(trifluoromethyl)benzyl)-1H-pyrrolo[2,3-b]pyridin-3-yl)-2-cyanoacrylic acid

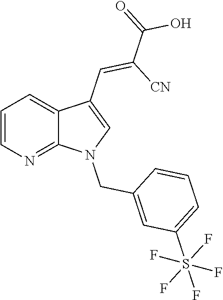 [SMILES: N#C/C(=C\c1cn(Cc2cccc(S(F)(F)(F)(F)F)c2)c2ncccc12)C(=O)O]

### (E)-2-cyano-3-(1-(3-(pentafluorosulfanyl)benzyl)-1H-pyrrolo[2,3-b]pyridin-3-yl)acrylic acid

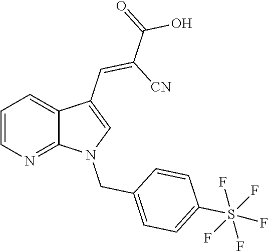 [SMILES: N#C/C(=C\c1cn(Cc2ccc(S(F)(F)(F)(F)F)cc2)c2ncccc12)C(=O)O]

### (E)-2-cyano-3-(1-(4-(pentafluorosulfanyl)benzyl)-1H-pyrrolo[2,3-b]pyridin-3-yl)acrylic acid

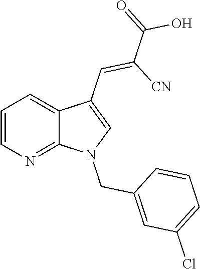 [SMILES: N#C/C(=C\c1cn(Cc2cccc(Cl)c2)c2ncccc12)C(=O)O]

### (E)-3-(1-(3-chlorobenzyl)-1H-pyrrolo[2,3-b]pyridin-3-yl)-2-cyanoacrylic acid

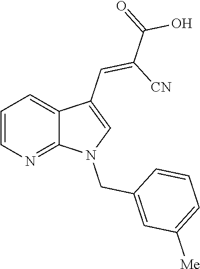 [SMILES: Cc1cccc(Cn2cc(/C=C(\C#N)C(=O)O)c3cccnc32)c1]

### (E)-2-cyano-3-(1-(3-methylbenzyl)-1H-pyrrolo[2,3-b]pyridin-3-yl)acrylic acid

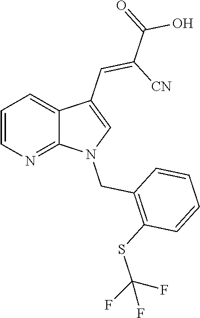 [SMILES: N#C/C(=C\c1cn(Cc2ccccc2SC(F)(F)F)c2ncccc12)C(=O)O]

### (E)-2-cyano-3-(1-(2-((trifluoromethyl)thio)benzyl)-1H-pyrrolo[2,3-b]pyridin-3-yl)acrylic acid

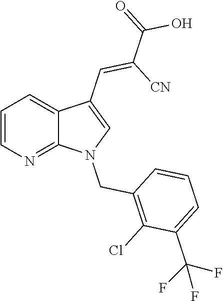 [SMILES: N#C/C(=C\c1cn(Cc2cccc(C(F)(F)F)c2Cl)c2ncccc12)C(=O)O]

### (E)-3-(1-(2-chloro-3-(trifluoromethyl)benzyl)-1H-pyrrolo[2,3-b]pyridin-3-yl)-2-cyanoacrylic acid

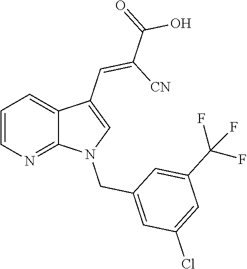 [SMILES: N#C/C(=C\c1cn(Cc2cc(Cl)cc(C(F)(F)F)c2)c2ncccc12)C(=O)O]

### (E)-3-(1-(3-chloro-5-(trifluoromethyl)benzyl)-1H-pyrrolo[2,3-b]pyridin-3-yl)-2-cyanoacrylic acid

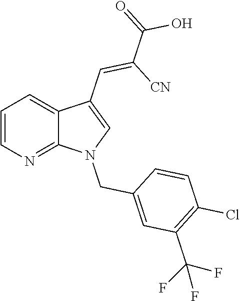 [SMILES: N#C/C(=C\c1cn(Cc2ccc(Cl)c(C(F)(F)F)c2)c2ncccc12)C(=O)O]

### (E)-3-(1-(4-chloro-3-(trifluoromethyl)benzyl)-1H-pyrrolo[2,3-b]pyridin-3-yl)-2-cyanoacrylic acid

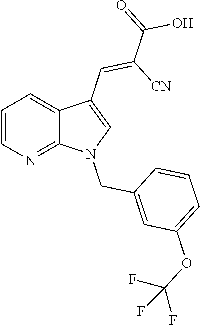 [SMILES: N#C/C(=C\c1cn(Cc2cccc(OC(F)(F)F)c2)c2ncccc12)C(=O)O]

### (E)-2-cyano-3-(1-(3-(trifluoromethoxy)benzyl)-1H-pyrrolo[2,3-b]pyridin-3-yl)acrylic acid

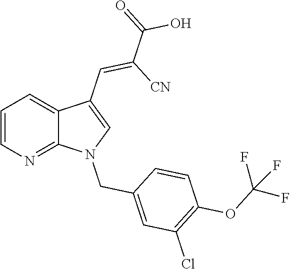 [SMILES: N#C/C(=C\c1cn(Cc2ccc(OC(F)(F)F)c(Cl)c2)c2ncccc12)C(=O)O]

### (E)-3-(1-(3-chloro-4-(trifluoromethoxy)benzyl)-1H-pyrrolo[2,3-b]pyridin-3-yl)-2-cyanoacrylic acid

An assay similar to that described in Example 16, pp. 43-46, of PCT/US2021/039502, filed Jun. 21, 2021 under the title “Composition and Methods for Modulating Hair Growth,” attorney docket number UCH-28325, was conducted on some of the compounds described herein. The IC₅₀ values at inhibiting mitochondrial oxidation for the tested compounds was less than 1 μM, and in some cases less than 100 nM.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A compound represented by a formula: 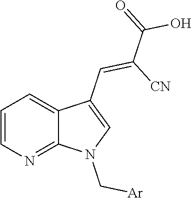
or a pharmaceutically acceptable salt thereof;
wherein Ar is optionally substituted aryl.

2. The compound of claim 1, which is:

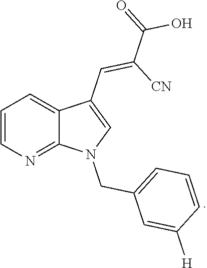 [SMILES: N#C/C(=C\c1cn(Cc2ccccc2)c2ncccc12)C(=O)O]

3. The compound of claim 1, which is:

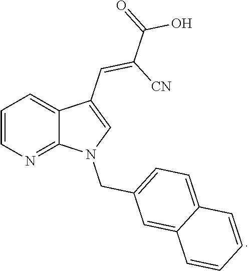 [SMILES: N#C/C(=C\c1cn(Cc2ccc3ccccc3c2)c2ncccc12)C(=O)O]

4. The compound of claim 1, which is:

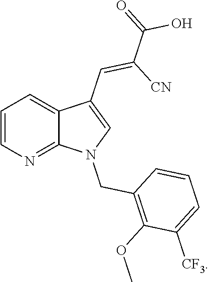 [SMILES: COc1c(Cn2cc(/C=C(\C#N)C(=O)O)c3cccnc32)cccc1C(F)(F)F]

5. The compound of claim 1, which is:

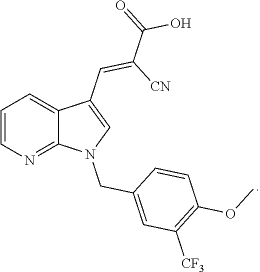 [SMILES: COc1ccc(Cn2cc(/C=C(\C#N)C(=O)O)c3cccnc32)cc1C(F)(F)F]

6. The compound of claim 1, which is:

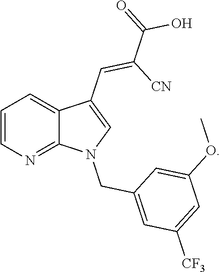 [SMILES: COc1cc(Cn2cc(/C=C(\C#N)C(=O)O)c3cccnc32)cc(C(F)(F)F)c1]

7. The compound of claim 1, which is:

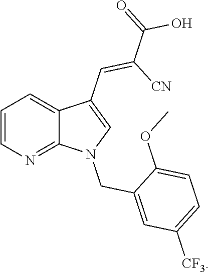 [SMILES: COc1ccc(C(F)(F)F)cc1Cn1cc(/C=C(\C#N)C(=O)O)c2cccnc21]

8. The compound of claim 1, which is:

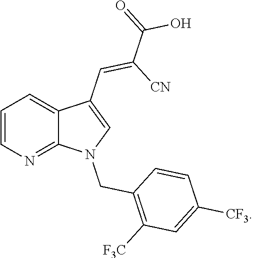 [SMILES: N#C/C(=C\c1cn(Cc2ccc(C(F)(F)F)cc2C(F)(F)F)c2ncccc12)C(=O)O]

9. The compound of claim 1, which is:

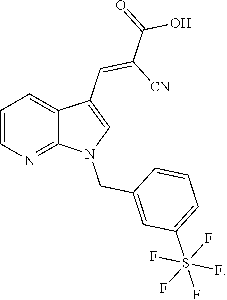 [SMILES: N#C/C(=C\c1cn(Cc2cccc(S(F)(F)(F)(F)F)c2)c2ncccc12)C(=O)O]

10. The compound of claim 1, which is:

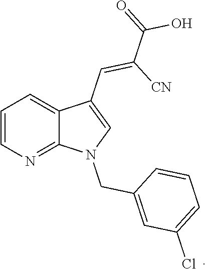 [SMILES: N#C/C(=C\c1cn(Cc2cccc(Cl)c2)c2ncccc12)C(=O)O]

11. The compound of claim 1, which is:

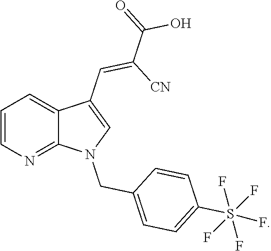 [SMILES: N#C/C(=C\c1cn(Cc2ccc(S(F)(F)(F)(F)F)cc2)c2ncccc12)C(=O)O]

12. The compound of claim 1, which is:

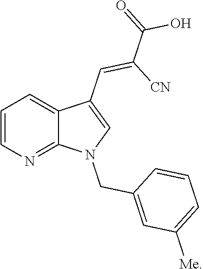 [SMILES: Cc1cccc(Cn2cc(/C=C(\C#N)C(=O)O)c3cccnc32)c1]

13. The compound of claim 1, which is:

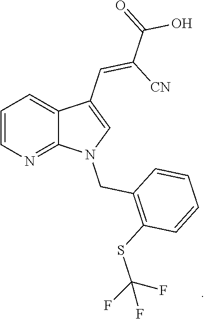 [SMILES: N#C/C(=C\c1cn(Cc2ccccc2SC(F)(F)F)c2ncccc12)C(=O)O]

14. The compound of claim 1, which is:

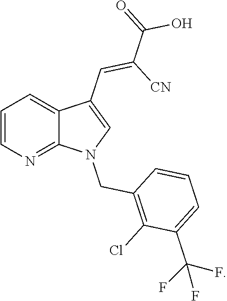 [SMILES: N#C/C(=C\c1cn(Cc2cccc(C(F)(F)F)c2Cl)c2ncccc12)C(=O)O]

15. The compound of claim 1, which is:

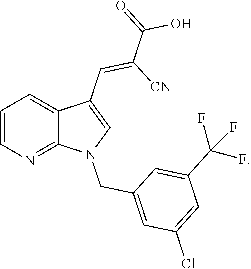 [SMILES: N#C/C(=C\c1cn(Cc2cc(Cl)cc(C(F)(F)F)c2)c2ncccc12)C(=O)O]

16. The compound of claim 1, which is:

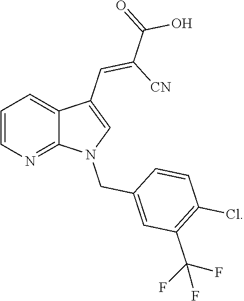 [SMILES: N#C/C(=C\c1cn(Cc2ccc(Cl)c(C(F)(F)F)c2)c2ncccc12)C(=O)O]

17. The compound of claim 1, which is:

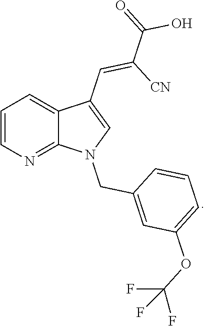 [SMILES: N#C/C(=C\c1cn(Cc2cccc(OC(F)(F)F)c2)c2ncccc12)C(=O)O]

18. The compound of claim 1, which is: C

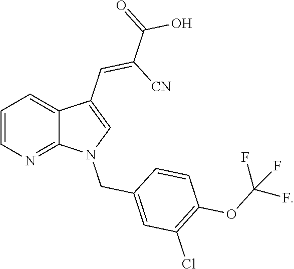 [SMILES: N#C/C(=C\c1cn(Cc2ccc(OC(F)(F)F)c(Cl)c2)c2ncccc12)C(=O)O]

19. A compound represented by a formula: 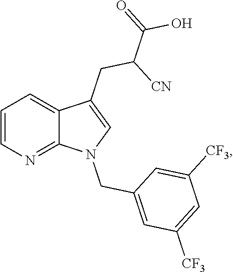 [SMILES: N#CC(Cc1cn(Cc2cc(C(F)(F)F)cc(C(F)(F)F)c2)c2ncccc12)C(=O)O]
or a pharmaceutically acceptable salt thereof.

20.-21. (canceled)

22. A method of growing hair, comprising: administering a compound of claim 1 to the skin of a mammal in the area where hair growth is intended.

23.-29. (canceled)
